(19) United States
(12) Patent Application Publication
(10) Pub. No.: US 2021/0179674 A1
(43) Pub. Date: Jun. 17, 2021

(54) YEAST WITH IMPROVED ALCOHOL PRODUCTION

(71) Applicant: DANISCO US INC, Palo Alto, CA (US)

(72) Inventors: Xiaochun Fan, West Chester, PA (US); Zhongqiang Chen, Wilmington, DE (US); Min Qi, Hockessin, DE (US)

(21) Appl. No.: 16/759,177

(22) PCT Filed: Oct. 22, 2018
(86) PCT No.: PCT/US18/56861

§ 371 Date: Apr. 24, 2020

Related U.S. Application Data
(60) Provisional application No. 62/576,444, filed on Oct. 24, 2017.

(51) Int. Cl. C07K 14/395; C12N 9/24; C12N 15/81

(52) U.S. Cl. CPC C07K 14/395; C12N 15/81; C12N 9/24

(57) ABSTRACT

Described are compositions and methods relating to yeast cells having a genetic mutation that give rise to increased alcohol production. Such yeast is well-suited for use in alcohol production to reduce fermentation time and/or increase yields.

### TECHNICAL FIELD

The present strains and methods relate to yeast having a genetic mutation that results in increased ethanol production. Such yeast is well-suited for use in alcohol production to reduce fermentation time and/or increase yields.

### BACKGROUND

Many countries make fuel alcohol from fermentable substrates, such as corn starch, sugar cane, cassava, and molasses. According to the Renewable Fuel Association (Washington D.C., United States), 2015 fuel ethanol production was close to 15 billion gallons in the United States, alone.

In view of the large amount of alcohol produced in the world, even a minor increase in the efficiency of a fermenting organism can result in a tremendous increase in the amount of available alcohol. Accordingly, the need exists for organisms that are more efficient at producing alcohol.

### SUMMARY

Described are methods relating to modified yeast cells capable of increased alcohol production. Aspects and embodiments of the compositions and methods are described in the following, independently-numbered paragraphs.

1. In one aspect, modified yeast cells derived from parental yeast cells are provided, the modified cells comprising a genetic alteration that causes the modified cells to produce a decreased amount of functional MNN4 polypeptide compared to the parental cells, wherein the modified cells produce during fermentation an increased amount of ethanol compared to parental cells under equivalent fermentation conditions.

2. In some embodiments of the modified cells of paragraph 1, the genetic alteration comprises a disruption of the YKL201C gene present in the parental cells.

3. In some embodiments of the modified cells of paragraph 2, disruption of the YKL201C gene is the result of deletion of all or part of the YKL201C gene.

4. In some embodiments of the modified cells of paragraph 2, disruption of the YKL201C gene is the result of deletion of a portion of genomic DNA comprising the YKL201C gene.

5. In some embodiments of the modified cells of paragraph 2, disruption of the YKL201C gene is the result of mutagenesis of the YKL201C gene.

6. In some embodiments of the modified cells of any of paragraphs 2-5, disruption of the YKL201C gene is performed in combination with introducing a gene of interest at the genetic locus of the YKL201C gene.

7. In some embodiments of the modified cells of any of paragraphs 1-6, the cells do not produce functional MNN4 polypeptides.

8. In some embodiments of the modified cells of any of paragraphs 1-6, the cells do not produce MNN4 polypeptides.

9. In some embodiments of the modified cells of any of paragraphs 1-8, the cells further comprise an exogenous gene encoding a carbohydrate processing enzyme.

10. In some embodiments, the modified cells of any of paragraphs 1-9 further comprise an alteration in the glycerol pathway and/or the acetyl-CoA pathway.

11. In some embodiments, the modified cells of any of paragraphs 1-10 further comprise an alternative pathway for making ethanol.

12. In some embodiments of the modified cells of any of paragraphs 1-11, the cells are of a Saccharomyces spp.

13. In another aspect, a method for producing a modified yeast cell is provided, comprising: introducing a genetic alteration into a parental yeast cell, which genetic alteration reduces or prevents the production of functional MNN4 polypeptide compared to the parental cells, thereby producing modified cells that produces during fermentation an increased amount of ethanol compared to the parental cells under equivalent fermentation conditions.

14. In some embodiments of the method of paragraph 13, the genetic alteration comprises disrupting the YKL201C gene in the parental cells by genetic manipulation.

15. In some embodiments of the method of paragraph 13 or 14, the genetic alteration comprises deleting the YKL201C gene in the parental cells using genetic manipulation.

16. In some embodiments of the method of any of paragraphs 13-15, disruption of the YKL201C gene is performed in combination with introducing a gene of interest at the genetic locus of the YKL201C gene.

17. In some embodiments of the method of any of paragraphs 13-16, disruption of the YKL201C gene is performed in combination with making an alteration in the glycerol pathway and/or the acetyl-CoA pathway.

18. In some embodiments of the method of any of paragraphs 13-17, disruption of the YKL201C gene is performed in combination with adding an alternative pathway for making ethanol.

19. In some embodiments of the method of any of paragraphs 13-18, disruption of the YKL201C gene is performed in combination with disrupting the YJL065 gene present in the parental cells.

20. In some embodiments of the method of any of paragraphs 13-19, disruption of the YKL201C gene is performed in combination with introducing an exogenous gene encoding a carbohydrate processing enzyme.

21. In some embodiments of the method of any of paragraphs 13-20, the modified cell is from a Saccharomyces spp.

22. In some embodiments of the method of any of paragraphs 13-21, the amount of ethanol produced by the modified yeast cells and the parental yeast cells is measured at 54 hours following inoculation of a hydrolyzed starch substrate comprising 34-35% dissolved solids and having a pH of 4.8.

23. In another aspect, modified yeast cells produced by the method of any of paragraphs 13-22 are provided.

These and other aspects and embodiments of present modified cells and methods will be apparent from the description.

### DETAILED DESCRIPTION

### I. Overview

The present compositions and methods relate to modified yeast cells demonstrating increased ethanol production compared to their parental cells. When used for ethanol production, the modified cells allow for increased yields and or shorter fermentation times, thereby increasing the supply of ethanol for world consumption.

### II. Definitions

Prior to describing the present strains and methods in detail, the following terms are defined for clarity. Terms not defined should be accorded their ordinary meanings as used in the relevant art.

As used herein, “alcohol” refer to an organic compound in which a hydroxyl functional group (—OH) is bound to a saturated carbon atom.

As used herein, “butanol” refers to the butanol isomers 1-butanol, 2-butanol, tert-butanol, and/or isobutanol (also known as 2-methyl-1-propanol) either individually or as mixtures thereof.

As used herein, “yeast cells” yeast strains, or simply “yeast” refer to organisms from the phyla Ascomycota and Basidiomycota. Exemplary yeast is budding yeast from the order Saccharomycetales. Particular examples of yeast are Saccharomyces spp., including but not limited to S. cerevisiae. Yeast include organisms used for the production of fuel alcohol as well as organisms used for the production of potable alcohol, including specialty and proprietary yeast strains used to make distinctive-tasting beers, wines, and other fermented beverages.

As used herein, the phrase “variant yeast cells,” “modified yeast cells,” or similar phrases (see above), refer to yeast that include genetic modifications and characteristics described herein. Variant/modified yeast do not include naturally occurring yeast.

As used herein, the phrase “substantially free of an activity,” or similar phrases, means that a specified activity is either undetectable in an admixture or present in an amount that would not interfere with the intended purpose of the admixture.

As used herein, the terms “polypeptide” and “protein” (and their respective plural forms) are used interchangeably to refer to polymers of any length comprising amino acid residues linked by peptide bonds. The conventional one-letter or three-letter codes for amino acid residues are used herein and all sequence are presented from an N-terminal to C-terminal direction. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins.” Such proteins can be derived from organisms of different genera and/or species, or even different classes of organisms (e.g., bacteria and fungi). Related proteins also encompass homologs determined by primary sequence analysis, determined by secondary or tertiary structure analysis, or determined by immunological cross-reactivity.

As used herein, the term “homologous protein” refers to a protein that has similar activity and/or structure to a reference protein. It is not intended that homologs necessarily be evolutionarily related. Thus, it is intended that the term encompass the same, similar, or corresponding enzyme(s) (i.e., in terms of structure and function) obtained from different organisms. In some embodiments, it is desirable to identify a homolog that has a quaternary, tertiary and/or primary structure similar to the reference protein. In some embodiments, homologous proteins induce similar immunological response(s) as a reference protein. In some embodiments, homologous proteins are engineered to produce enzymes with desired activity(ies).

The degree of homology between sequences can be determined using any suitable method known in the art (see, e.g., Smith and Waterman (1981) Adv. Appl. Math. 2:482; Needleman and Wunsch (1970) J. Mol. Biol., 48:443; Pearson and Lipman (1988) Proc. Natl. Acad. Sci. USA 85:2444; programs such as GAP, BESTFIT, FASTA, and TFASTA in the Wisconsin Genetics Software Package (Genetics Computer Group, Madison, Wis.); and Devereux et al. (1984) Nucleic Acids Res. 12:387-95).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle, (Feng and Doolittle (1987) J. Mol. Evol. 35:351-60). The method is similar to that described by Higgins and Sharp ((1989) CABIOS 5:151-53). Useful PILEUP parameters including a default gap weight of 3.00, a default gap length weight of 0.10, and weighted end gaps. Another example of a useful algorithm is the BLAST algorithm, described by Altschul et al. ((1990) J. Mol. Biol. 215:403-10) and Karlin et al. ((1993) Proc. Natl. Acad. Sci. USA 90:5873-87). One particularly useful BLAST program is the WU-BLAST-2 program (see, e.g., Altschul et al. (1996) Meth. Enzymol. 266:460-80). Parameters “W,” “T,” and “X” determine the sensitivity and speed of the alignment. The BLAST program uses as defaults a word-length (W) of 11, the BLOSUM62 scoring matrix (see, e.g., Henikoff and Henikoff (1989) Proc. Natl. Acad. Sci. USA 89:10915) alignments (B) of 50, expectation (E) of 10, M′5, N′-4, and a comparison of both strands.

As used herein, the phrases “substantially similar” and “substantially identical,” in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Percent sequence identity is calculated using CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF.

Another indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, the term “gene” is synonymous with the term “allele” in referring to a nucleic acid that encodes and directs the expression of a protein or RNA. Vegetative forms of filamentous fungi are generally haploid, therefore a single copy of a specified gene (i.e., a single allele) is sufficient to confer a specified phenotype.

As used herein, the terms “wild-type” and “native” are used interchangeably and refer to genes proteins or strains found in nature.

As used herein, the term “protein of interest” refers to a polypeptide that is desired to be expressed in modified yeast. Such a protein can be an enzyme, a substrate-binding protein, a surface-active protein, a structural protein, a selectable marker, or the like, and can be expressed at high levels. The protein of interest is encoded by a modified endogenous gene or a heterologous gene (i.e., gene of interest”) relative to the parental strain. The protein of interest can be expressed intracellularly or as a secreted protein.

As used herein, “deletion of a gene,” refers to its removal from the genome of a host cell. Where a gene includes control elements (e.g., enhancer elements) that are not located immediately adjacent to the coding sequence of a gene, deletion of a gene refers to the deletion of the coding sequence, and optionally adjacent enhancer elements, including but not limited to, for example, promoter and/or terminator sequences, but does not require the deletion of non-adjacent control elements.

As used herein, “disruption of a gene” refers broadly to any genetic or chemical manipulation, i.e., mutation, that substantially prevents a cell from producing a functional gene product, e.g., a protein, in a host cell. Exemplary methods of disruption include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and combinations and variations, thereof, any of which mutations substantially prevent the production of a function gene product. A gene can also be disrupted using RNAi, antisense, or any other method that abolishes gene expression. A gene can be disrupted by deletion or genetic manipulation of non-adjacent control elements.

As used herein, the terms “genetic manipulation” and “genetic alteration” are used interchangeably and refer to the alteration/change of a nucleic acid sequence. The alteration can include but is not limited to a substitution, deletion, insertion or chemical modification of at least one nucleic acid in the nucleic acid sequence.

As used herein, a “primarily genetic determinant” refers to a gene, or genetic manipulation thereof, that is necessary and sufficient to confer a specified phenotype in the absence of other genes, or genetic manipulations, thereof. However, that a particular gene is necessary and sufficient to confer a specified phenotype does not exclude the possibility that additional effects to the phenotype can be achieved by further genetic manipulations.

As used herein, a “functional polypeptide/protein” is a protein that possesses an activity, such as an enzymatic activity, a binding activity, a surface-active property, or the like, and which has not been mutagenized, truncated, or otherwise modified to abolish or reduce that activity. Functional polypeptides can be thermostable or thermolabile, as specified.

As used herein, “a functional gene” is a gene capable of being used by cellular components to produce an active gene product, typically a protein. Functional genes are the antithesis of disrupted genes, which are modified such that they cannot be used by cellular components to produce an active gene product, or have a reduced ability to be used by cellular components to produce an active gene product.

As used herein, yeast cells have been “modified to prevent the production of a specified protein” if they have been genetically or chemically altered to prevent the production of a functional protein/polypeptide that exhibits an activity characteristic of the wild-type protein. Such modifications include, but are not limited to, deletion or disruption of the gene encoding the protein (as described, herein), modification of the gene such that the encoded polypeptide lacks the aforementioned activity, modification of the gene to affect post-translational processing or stability, and combinations, thereof.

As used herein, “attenuation of a pathway” or “attenuation of the flux through a pathway” i.e., a biochemical pathway, refers broadly to any genetic or chemical manipulation that reduces or completely stops the flux of biochemical substrates or intermediates through a metabolic pathway. Attenuation of a pathway may be achieved by a variety of well-known methods. Such methods include but are not limited to: complete or partial deletion of one or more genes, replacing wild-type alleles of these genes with mutant forms encoding enzymes with reduced catalytic activity or increased Km values, modifying the promoters or other regulatory elements that control the expression of one or more genes, engineering the enzymes or the mRNA encoding these enzymes for a decreased stability, misdirecting enzymes to cellular compartments where they are less likely to interact with substrate and intermediates, the use of interfering RNA, and the like.

As used herein, “aerobic fermentation” refers to growth in the presence of oxygen.

As used herein, “anaerobic fermentation” refers to growth in the absence of oxygen.

As used herein, the singular articles “a,” “an,” and “the” encompass the plural referents unless the context clearly dictates otherwise. All references cited herein are hereby incorporated by reference in their entirety. The following abbreviations/acronyms have the following meanings unless otherwise specified:
° C. degrees Centigrade
AA α-amylase
bp base pairs
DNA deoxyribonucleic acid
DP degree of polymerization
ds or DS dry solids
EtOH ethanol
g or gm gram
g/L grams per liter
GA glucoamylase
GAU/g ds glucoamylase units per gram dry solids
H₂O water
HPLC high performance liquid chromatography
hr or h hour
kg kilogram
M molar
mg milligram
mL or ml milliliter
ml/min milliliter per minute
mM millimolar
N normal
nm nanometer
PCR polymerase chain reaction
ppm parts per million
SAPU/g ds protease units per gram dry solids
SSCU/g ds fungal alpha-amylase units per gram dry solids
Δ relating to a deletion
microgram
μL and μl microliter
μM micromolar

### III. Modified Yeast Cells Having Reduced or Eliminated MNN4 Activity

In one aspect, modified yeast cells are provided, the modified yeast having a genetic alteration that causes the cells of the modified strain to produce a decreased amount of functional mannosylphosphate transferase 4 (MNN4) polypeptide, encoded by the YKL201C gene, compared to the otherwise-identical parental cells. MNN4 is a 1,178-amino acid protein required for phosphorylation of N-linked oligosaccharides in Saccharomyces cerevisiae. MNN4 has the topology of type II membrane proteins features a repeated sequence of lysine and glutamic acid at the C-terminus. Manipulation of MNN4 expression leads to altered mannosylphosphate content in cell wall mannans (Odani, T. et al. (1996) Glycobiology 6:805-10).

Applicants have discovered that yeast having a genetic alteration that affects MNN4 function demonstrate increased ethanol production in fermentations, allowing for higher yields, shorter fermentation times or both. Shorter fermentation times allow alcohol production facilities to run more fermentation in a given period of time, increasing productivity.

The reduction in the amount of functional MNN4 protein can result from disruption of the YKL201C gene present in the parental strain. Because disruption of the YKL201C gene is a primary genetic determinant for conferring the increased ethanol-production-phenotype to the modified cells, in some embodiments the modified cells need only comprise a disrupted YKL201C gene, while all other genes can remain intact. In other embodiments, the modified cells can optionally include additional genetic alterations compared to the parental cells from which they are derived. While such additional genetic alterations are not necessary to confer the described phenotype, they may confer other advantages to the modified cells.

Disruption of the YKL201C gene can be performed using any suitable methods that substantially prevent expression of a function YKL201C gene product, i.e., MNN4. Exemplary methods of disruption as are known to one of skill in the art include but are not limited to: complete or partial deletion of the YKL201C gene, including complete or partial deletion of, e.g., the MNN4-coding sequence, the promoter, the terminator, an enhancer, or another regulatory element; and complete or partial deletion of a portion of the chromosome that includes any portion of the YKL201C gene. Particular methods of disrupting the YKL201C gene include making nucleotide substitutions or insertions in any portion of the YKL201C gene, e.g., the MNN4-coding sequence, the promoter, the terminator, an enhancer, or another regulatory element. Preferably, deletions, insertions, and/or substitutions (collectively referred to as mutations) are made by genetic manipulation using sequence-specific molecular biology techniques, as opposed to by chemical mutagenesis, which is generally not targeted to specific nucleic acid sequences. Nonetheless, chemical mutagenesis can, in theory, be used to disrupt the YKL201C gene.

Mutations in the YKL201C gene can reduce the efficiency of the YKL201C promoter, reduce the efficiency of a YKL201C enhancer, interfere with the splicing or editing of the YKL201C mRNA, interfere with the translation of the YKL201C mRNA, introduce a stop codon into the MNN4-coding sequence to prevent the translation of full-length MNN4 protein, change the coding sequence of the MNN4 protein to produce a less active or inactive protein or reduce MNN4 interaction with other nuclear protein components, or DNA, change the coding sequence of the MNN4 protein to produce a less stable protein or target the protein for destruction, cause the MNN4 protein to misfold or be incorrectly modified (e.g., by glycosylation), or interfere with cellular trafficking of the MNN4 protein. In some embodiments, these and other genetic manipulations act to reduce or prevent the expression of a functional MNN4 protein, or reduce or prevent the normal biological activity of MNN4.

In some embodiments, the present modified cells include genetic manipulations that reduce or prevent the expression of a functional MNN4 protein, or reduce or prevent the normal biological activity of MNN4.

In some embodiments, the decrease in the amount of functional MNN4 polypeptide in the modified cells is a decrease of at least 30%, at least 40%, at least 50%, at least 60%, at least 70%, at least 80%, at least 90%, at least 95%, at least 98%, at least 99%, or more, compared to the amount of functional MNN4 polypeptide in parental cells growing under the same conditions. In some embodiments, the reduction of expression of functional MNN4 protein in the modified cells is a reduction of at least 30%, at least 40%, at least 50%, at least 60%, at least 70%, at least 80%, at least 90%, at least 95%, at least 98%, at least 99%, or more, compared to the amount of functional MNN4 polypeptide in parental cells growing under the same conditions.

In some embodiments, the increase in alcohol in the modified cells is an increase of at least 1.0%, at least 1.1%, at least 1.2%, at least 1.3%, at least 1.4%, at least 1.5%, at least 1.6%, at least 1.7%, at least 1.8%, at least 1.9%, or even at least at 2%, or more, compared to the amount of alcohol produced in parental cells growing under the same conditions.

Preferably, disruption of the YKL201C gene is performed by genetic manipulation using sequence-specific molecular biology techniques, as opposed to chemical mutagenesis, which is generally not targeted to specific nucleic acid sequences. However, chemical mutagenesis is not excluded as a method for making modified yeast cells.

In some embodiments, the parental cell that is modified already includes a gene of interest, such as a gene encoding a selectable marker, carbohydrate-processing enzyme, or other polypeptide. In some embodiments, a gene of introduced is subsequently introduced into the modified cells.

The amino acid sequence of the exemplified S. cerevisiae MNN4 polypeptide is shown, below, as SEQ ID NO: 1:

MLQRISSKLH RRFLSGLLRV KHYPLRRILL PLILLQIIII
TFIWSNSPQR NGLGRDADYL LPNYNELDSD DDSWYSILTS
SFKNDRKIQF AKTLYENLKF GTNPKWVNEY TLQNDLLSVK
MGPRKGSKLE SVDELKFYDF DPRLTWSVVL NHLQNNDADQ
PEKLPFSWYD WTTFHELNKL ISIDKTVLPC NFLFQSAFDK
ESLEAIETEL GEPLFLYERP KYAQKLWYKA ARNQDRIKDS
KELKKHCSKL FTPDGHGSPK GLRFNTQFQI KELYDKVRPE
VYQLQARNYI LTTQSHPLSI SIIESDNSTY QVPLQTEKSK
NLVQSGLLQE YINDNINSTN KRKKNKQDVE FNHNRLFQEF
VNNDQVNSLY KLEIEETDKF TFDKDLVYLS PSDFKFDASK
KIEELEEQKK LYPDKFSAHN ENYLNSLKNS VKTSPALQRK
FFYEAGAVKQ YKGMGFHRDK RFFNVDTLIN DKQEYQARLN
SMIRTFQKFT KANGIISWLS HGTLYGYLYN GMAFPWDNDF
DLQMPIKHLQ LLSQYFNQSL ILEDPRQGNG RYFLDVSDSL
TVRINGNGKN NIDARFIDVD TGLYIDITGL ASTSAPSRDY
LNSYIEERLQ EEHLDINNIP ESNGETATLP DKVDDGLVNM
ATLNITELRD YITSDENKNH KRVPTDTDLK DLLKKELEEL
PKSKTIENKL NPKQRYFLNE KLKLYNCRNN HFNSFEELSP
LINTVFHGVP ALIPHRHTYC LHNEYHVPDR YAFDAYKNTA
YLPEFRFWFD YDGLKKCSNI NSWYPNIPSI NSWNPNLLKE
ISSTKFESKL FDSNKVSEYS FKNLSMDDVR LIYKNIPKAG
FIEVFTNLYN SFNVTAYRQK ELEIQYCQNL TFIEKKKLLH
QLRINVAPKL SSPAKDPFLF GYEKAMWKDL SKSMNQTTLD
QVTKIVHEEY VGKIIDLSES LKYRNFSLFN ITFDETGTTL
DDNTEDYTPA NTVEVNPVDF KSNLNFSSNS FLDLNSYGLD
LFAPTLSDVN RKGIQMFDKD PIIVYEDYAY AKLLEERKRR
EKKKKEEEEK KKKEEEEKKK KEEEEKKKKE EEEKKKKEEE
EKKKKEEEEK KKQEEEEKKK KEEEEKKKQE EGEKMKNEDE
ENKKNEDEEK KKNEEEEKKK QEEKNKKNED EEKKKQEEEE
KKKNEEEEKK KQEEGHSN

Based on a BLAST search of the NCBI protein database, the described MNN4 polypeptide is 100% identical to at least 30 deposits in GenBank.

It is expected that the present compositions and methods are applicable to other structurally similar MNN4 polypeptides, as well as other related proteins, homologs, and functionally similar polypeptides.

In some embodiments of the present compositions and methods, the amino acid sequence of the MNN4 protein that is altered in production levels has a specified degree of overall amino acid sequence identity to the amino acid sequence of SEQ ID NO: 1, e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99% identity, to SEQ ID NO: 1.

In some embodiments of the present compositions and methods, the YKL201C gene that is disrupted encodes a MNN4 protein that has a specified degree of overall amino acid sequence identity to the amino acid sequence of SEQ ID NO: 1, e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99% identity, to SEQ ID NO: 1.

The amino acid sequence information provided, herein, readily allows the skilled person to identify a MNN4 protein, and the nucleic acid sequence encoding a MNN4 protein, in any yeast, and to make appropriate disruptions in the YKL201C gene to affect the production of the MNN4 protein.

In some embodiments, the decrease in the amount of functional MNN4 polypeptide in the modified cells is a decrease of at least 30%, at least 40%, at least 50%, at least 60%, at least 70%, at least 80%, at least 90%, at least 95%, at least 98%, at least 99%, or more, compared to the amount of functional MNN4 polypeptide in parental cells growing under the same conditions. In some embodiments, the reduction of expression of functional MNN4 protein in the modified cells is a reduction of at least 30%, at least 40%, at least 50%, at least 60%, at least 70%, at least 80%, at least 90%, at least 95%, at least 98%, at least 99%, or more, compared to the amount of functional MNN4 polypeptide in parental cells growing under the same conditions.

In some embodiments, the increase in ethanol production by the modified cells, compared to otherwise identical parental cells, is an increase of at least 0.2%, at least 0.4%, at least 0.6%, at least 0.8%, at least 1.0%, at least 1.2%, at least 1.4%, at least 1.6%, at least 1.8%, at least 2.0% or more.

### IV. Combination of Decreased MNN4 Expression with Other Mutations that Affect Alcohol Production

In some embodiments, in addition to expressing decreased amounts of functional MNN4 polypeptides, the present modified yeast cells further include additional modifications that affect ethanol production.

In particular embodiments the modified yeast cells include an artificial or alternative pathway resulting from the introduction of a heterologous phosphoketolase gene and a heterologous phosphotransacetylase gene. An exemplary phosphoketolase can be obtained from Gardnerella vaginalis (UniProt/TrEMBL Accession No.: WP_016786789). An exemplary phosphotransacetylase can be obtained from Lactobacillus plantarum (UniProt/TrEMBL Accession No.: WP_003641060).

The modified cells may further include mutations that result in attenuation of the native glycerol biosynthesis pathway, which are known to increase alcohol production. Methods for attenuation of the glycerol biosynthesis pathway in yeast are known and include reduction or elimination of endogenous NAD-dependent glycerol 3-phosphate dehydrogenase (GPD) or glycerol phosphate phosphatase activity (GPP), for example by disruption of one or more of the genes GPD1, GPD2, GPP1 and/or GPP2. See, e.g., U.S. Pat. No. 9,175,270 (Elke et al.), U.S. Pat. No. 8,795,998 (Pronk et al.) and U.S. Pat. No. 8,956,851 (Argyros et al.).

The modified yeast may further feature increased acetyl-CoA synthase (also referred to acetyl-CoA ligase) activity (EC 6.2.1.1) to scavenge (i.e., capture) acetate produced by chemical or enzymatic hydrolysis of acetyl-phosphate (or present in the culture medium of the yeast for any other reason) and converts it to Ac-CoA. This avoids the undesirable effect of acetate on the growth of yeast cells and may further contribute to an improvement in alcohol yield. Increasing acetyl-CoA synthase activity may be accomplished by introducing a heterologous acetyl-CoA synthase gene into cells, increasing the expression of an endogenous acetyl-CoA synthase gene and the like. A particularly useful acetyl-CoA synthase for introduction into cells can be obtained from Methanosaeta concilii (UniProt/TrEMBL Accession No.: WP_013718460). Homologs of this enzymes, including enzymes having at least 85%, at least 90%, at least 92%, at least 95%, at least 97%, at least 98% and even at least 99% amino acid sequence identity to the aforementioned acetyl-CoA synthase from Methanosaeta concilii, are also useful in the present compositions and methods.

In some embodiments the modified cells may further include a heterologous gene encoding a protein with NAD⁺-dependent acetylating acetaldehyde dehydrogenase activity and/or a heterologous gene encoding a pyruvate-formate lyase. The introduction of such genes in combination with attenuation of the glycerol pathway is described, e.g., in U.S. Pat. No. 8,795,998 (Pronk et al.). In some embodiments of the present compositions and methods the yeast expressly lack a heterologous gene(s) encoding an acetylating acetaldehyde dehydrogenase, a pyruvate-formate lyase or both.

In some embodiments, the present modified yeast cells further comprise a butanol biosynthetic pathway. In some embodiments, the butanol biosynthetic pathway is an isobutanol biosynthetic pathway. In some embodiments, the isobutanol biosynthetic pathway comprises a polynucleotide encoding a polypeptide that catalyzes a substrate to product conversion selected from the group consisting of: (a) pyruvate to acetolactate; (b) acetolactate to 2,3-dihydroxyisovalerate; (c) 2,3-dihydroxyisovalerate to 2-ketoisovalerate; (d) 2-ketoisovalerate to isobutyraldehyde; and (e) isobutyraldehyde to isobutanol. In some embodiments, the isobutanol biosynthetic pathway comprises polynucleotides encoding polypeptides having acetolactate synthase, keto acid reductoisomerase, dihydroxy acid dehydratase, ketoisovalerate decarboxylase, and alcohol dehydrogenase activity.

In some embodiments, the modified yeast cells comprising a butanol biosynthetic pathway further comprise a modification in a polynucleotide encoding a polypeptide having pyruvate decarboxylase activity. In some embodiments, the yeast cells comprise a deletion, mutation, and/or substitution in an endogenous polynucleotide encoding a polypeptide having pyruvate decarboxylase activity. In some embodiments, the polypeptide having pyruvate decarboxylase activity is selected from the group consisting of: PDC1, PDC5, PDC6, and combinations thereof. In some embodiments, the yeast cells further comprise a deletion, mutation, and/or substitution in one or more endogenous polynucleotides encoding FRA2, ALD6, ADH1, GPD2, BDH1, and YMR226C.

### V. Combination of Decreased MNN4 Expression with Other Beneficial Mutations

In some embodiments, in addition to expressing reduced amounts of MNN4 polypeptides, optionally in combination with other genetic modifications that benefit alcohol production, the present modified yeast cells further include any number of additional genes of interest encoding proteins of interest. Additional genes of interest may be introduced before, during, or after genetic manipulations that result in reduced expression of MNN4 polypeptides. Proteins of interest, include selectable markers, carbohydrate-processing enzymes, and other commercially-relevant polypeptides, including but not limited to an enzyme selected from the group consisting of a dehydrogenase, a transketolase, a phosphoketolase, a transladolase, an epimerase, a phytase, a xylanase, a β-glucanase, a phosphatase, a protease, an α-amylase, a β-amylase, a glucoamylase, a pullulanase, an isoamylase, a cellulase, a trehalase, a lipase, a pectinase, a polyesterase, a cutinase, an oxidase, a transferase, a reductase, a hemicellulase, a mannanase, an esterase, an isomerase, a pectinases, a lactase, a peroxidase and a laccase. Proteins of interest may be secreted, glycosylated, and otherwise-modified.

### VI. Yeast Cells Suitable for Modification

Yeasts are unicellular eukaryotic microorganisms classified as members of the fungus kingdom and include organisms from the phyla Ascomycota and Basidiomycota. Yeast that can be used for alcohol production include, but are not limited to, Saccharomyces spp., including S. cerevisiae, as well as Kluyveromyces, Lachancea and Schizosaccharomyces spp. Numerous yeast strains are commercially available, many of which have been selected or genetically engineered for desired characteristics, such as high alcohol production, rapid growth rate, and the like. Some yeasts have been genetically engineered to produce heterologous enzymes, such as glucoamylase or α-amylase.

### VII. Substrates and Products

Alcohol production from a number of carbohydrate substrates, including but not limited to corn starch, sugar cane, cassava, and molasses, is well known, as are innumerable variations and improvements to enzymatic and chemical conditions and mechanical processes. The present compositions and methods are believed to be fully compatible with such substrates and conditions.

Alcohol fermentation products include organic compound having a hydroxyl functional group (—OH) is bound to a carbon atom. Exemplary alcohols include but are not limited to methanol, ethanol, n-propanol, isopropanol, n-butanol, isobutanol, n-pentanol, 2-pentanol, isopentanol, and higher alcohols. The most commonly made fuel alcohols are ethanol, and butanol.

These and other aspects and embodiments of the present strains and methods will be apparent to the skilled person in view of the present description. The following examples are intended to further illustrate, but not limit, the strains and methods.

### EXAMPLES

### Example 1. Disruption of YKL201C in Saccharomyces cerevisiae

Genetic screening was performed to identify S. cerevisiae mutants with increased ethanol production, and a number of candidate genes were identified and selected for further testing (data not shown). One of the genes selected for further analysis was YKL201C, which encodes MNN4. The nucleotide sequence of the YKL201C gene is provided below as SEQ ID NO: 2:

ATGCTTCAGCGAATATCATCTAAACTTCACAGGCGGTTCTTATCTGGCCT
GCTGCGTGTCAAGCACTACCCATTAAGGCGCATTCTCCTTCCACTGATTC
TACTGCAGATCATCATTATAACGTTTATCTGGTCAAATTCACCGCAGCGT
AACGGACTTGGGCGGGACGCTGATTACCTTCTACCAAATTACAACGAACT
TGACAGTGATGATGATTCCTGGTATAGCATCCTGACTTCGTCTTTCAAAA
ACGATCGCAAGATCCAGTTCGCTAAGACATTATACGAAAATTTAAAATTC
GGCACCAACCCTAAATGGGTCAATGAATATACTCTGCAAAATGACCTGCT
CTCGGTCAAAATGGGCCCTCGAAAGGGCAGTAAGCTCGAATCCGTGGATG
AGTTGAAGTTTTACGACTTCGACCCTCGTCTCACGTGGTCCGTTGTGCTG
AACCATTTGCAAAATAATGACGCAGATCAGCCAGAAAAGTTACCCTTTTC
ATGGTACGACTGGACAACCTTCCACGAGCTGAATAAGCTGATTTCCATAG
ATAAAACTGTTCTGCCCTGCAATTTTCTTTTCCAGTCCGCTTTCGACAAA
GAGTCTTTAGAGGCCATTGAGACAGAGCTCGGCGAACCTTTGTTCCTATA
CGAAAGACCAAAGTACGCGCAGAAACTGTGGTACAAGGCCGCTAGAAACC
AGGACAGAATCAAAGACTCAAAGGAACTAAAAAAGCATTGTTCCAAGCTA
TTCACTCCAGACGGGCATGGCTCTCCTAAGGGTTTAAGATTTAATACGCA
ATTTCAAATAAAGGAGCTGTATGATAAAGTTAGACCCGAAGTTTACCAAT
TGCAGGCAAGAAACTACATTTTGACTACACAGTCGCATCCACTATCCATT
TCCATCATCGAATCAGATAATTCCACGTATCAAGTCCCCTTGCAAACTGA
AAAATCAAAAAACTTGGTGCAATCCGGCCTGTTGCAGGAATATATTAATG
ATAACATTAATTCTACGAACAAGAGAAAGAAAAATAAACAGGACGTAGAA
TTCAACCATAACAGGCTTTTCCAGGAATTCGTCAATAACGACCAAGTTAA
CTCCCTATACAAACTGGAAATTGAAGAAACTGATAAATTCACTTTTGATA
AAGATTTGGTTTATTTATCCCCTTCGGATTTCAAGTTCGATGCCTCCAAA
AAAATTGAAGAGTTAGAGGAACAGAAGAAACTCTATCCGGACAAATTTTC
CGCTCATAATGAGAATTATCTGAACAGTTTGAAGAATTCCGTAAAGACAA
GCCCTGCATTGCAAAGAAAGTTCTTCTATGAGGCTGGTGCCGTGAAGCAA
TATAAAGGTATGGGGTTCCATCGTGACAAGAGGTTCTTCAATGTTGATAC
ATTAATCAATGATAAACAAGAATACCAGGCTAGATTGAACTCAATGATCA
GAACATTCCAAAAGTTTACTAAAGCCAACGGCATCATATCTTGGTTGTCT
CACGGAACGCTGTACGGCTATCTTTACAATGGAATGGCTTTCCCTTGGGA
TAACGATTTCGACTTGCAAATGCCCATTAAGCATTTACAATTGCTCAGTC
AATACTTCAACCAATCTCTTATATTGGAAGACCCAAGACAGGGTAATGGA
CGTTATTTCCTAGACGTCAGCGACTCCTTGACAGTAAGAATTAACGGTAA
CGGTAAAAACAATATCGATGCAAGATTCATTGACGTCGACACCGGCCTTT
ACATTGATATTACCGGTCTAGCTAGCACTTCTGCCCCTAGTAGGGATTAC
TTGAATTCTTATATTGAAGAGCGGTTGCAAGAGGAACATTTGGATATCAA
TAATATCCCTGAATCGAACGGTGAGACCGCTACTTTGCCCGACAAAGTAG
ATGATGGGTTAGTCAATATGGCTACACTAAACATCACTGAGCTACGTGAT
TACATTACCAGCGACGAAAATAAAAATCATAAAAGAGTCCCCACTGATAC
TGATTTGAAAGATCTTTTGAAAAAGGAACTGGAAGAGTTACCAAAGTCTA
AGACCATTGAAAACAAGTTGAATCCTAAACAAAGATATTTTCTCAACGAA
AAACTTAAACTTTACAATTGTAGAAACAACCATTTTAACTCGTTCGAGGA
ACTATCTCCCTTAATCAATACTGTTTTCCATGGTGTGCCAGCGTTGATTC
CTCACAGACATACCTACTGCTTGCACAATGAATATCATGTACCTGATAGA
TATGCATTTGATGCTTACAAAAATACTGCTTATTTGCCCGAATTTAGATT
TTGGTTCGACTATGACGGGTTAAAGAAATGCAGTAATATTAATTCATGGT
ATCCAAACATCCCCAGTATTAATTCATGGAATCCGAACCTCTTGAAAGAA
ATATCGTCTACGAAATTTGAGTCGAAACTTTTTGATTCCAACAAAGTCTC
TGAATACTCTTTCAAAAACCTATCCATGGATGATGTTCGCTTAATTTATA
AAAATATTCCAAAAGCTGGCTTTATCGAGGTATTTACTAACTTGTACAAT
TCCTTCAATGTCACTGCATATAGGCAAAAGGAATTGGAAATTCAATACTG
CCAAAACCTGACATTTATTGAAAAAAAGAAATTATTACATCAATTGCGCA
TTAATGTTGCTCCTAAGTTAAGCTCCCCTGCAAAGGACCCATTTCTTTTT
GGTTATGAAAAAGCTATGTGGAAGGATTTATCAAAATCTATGAACCAGAC
TACATTAGATCAAGTTACCAAGATTGTTCATGAAGAATATGTCGGAAAAA
TTATTGATCTGTCCGAAAGTTTGAAATACAGGAATTTTTCACTTTTCAAC
ATTACTTTTGATGAAACTGGAACAACTCTAGATGATAACACAGAAGATTA
TACTCCTGCTAATACTGTTGAAGTAAATCCTGTGGATTTTAAATCAAATT
TAAACTTTAGTAGCAACTCCTTTTTGGATTTAAATTCATATGGTTTAGAC
CTTTTTGCGCCAACTTTATCCGACGTTAACAGAAAGGGTATTCAAATGTT
TGATAAGGACCCTATTATTGTATACGAGGACTATGCTTATGCCAAGTTAC
TTGAAGAAAGAAAGCGGAGGGAGAAGAAGAAGAAGGAGGAAGAGGAGAAG
AAGAAGAAGGAAGAAGAGGAAAAGAAGAAGAAGGAAGAAGAAGAAAAGAA
AAAGAAGGAAGAGGAAGAGAAGAAAAAGAAGGAAGAAGAAGAGAAGAAAA
AGAAGGAAGAAGAAGAAAAGAAGAAGCAGGAGGAAGAGGAGAAAAAGAAG
AAGGAAGAAGAAGAGAAGAAGAAGCAGGAAGAAGGAGAAAAGATGAAGAA
TGAAGATGAAGAAAATAAGAAGAATGAAGATGAAGAAAAGAAGAAGAACG
AAGAAGAGGAAAAAAAGAAGCAGGAAGAGAAAAACAAGAAGAATGAAGAT
GAAGAAAAGAAGAAGCAGGAAGAGGAAGAAAAGAAGAAGAACGAAGAAGA
GGAAAAAAAGAAGCAGGAGGAGGGGCACAGCAATTAA

The amino acid sequence is provided as SEQ ID NO: 1, above. Disruption of the YKL201C gene in S. cerevisiae was performed using standard molecular biology techniques by deleting essentially the entire coding region for MNN4 in the YKL201C gene. The host yeast used to make the modified yeast cells was commercially available FERMAX™ GOLD (Martrex, Inc., Chaska, Minn., US; herein “FG”). Deletion of the YKL021C gene was confirmed by colony PCR. The modified yeast was grown on non-selective media to remove the plasmid conferring kanamycin resistance used to select transformants. One of modified strains, designed FG-mnn4, was selected for further study.

### Example 2. Ethanol Production by Modified Yeast with Reduced Expression of MNN4

FG-mnn4 yeast harboring the deletion of the YKL201C gene was tested for its ability to produced ethanol compared to benchmark yeast (i.e., FERMAX™ GOLD), which is wild-type for the YKL201C gene) in liquefact (i.e., ground corn slurry having a dry solid (ds) value of 34.7%) prepared by adding 600 ppm urea, 0.124 SAPU/g ds FERMGEN™ 2.5× (an acid fungal protease), 0.33 GAU/g ds of a Trichoderma reesei glucoamylase variant and 1.46 SSCU/g ds Aspergillus kawachii α-amylase, at pH 4.8.

5 ml (about 5.5 grams) of liquefact was apportioned to 20 ml glass vials and inoculated with fresh overnight cultures from colonies of the modified strain or FG strain at 32° C. Samples from three FG-mnn4 mutants and four FG parental yeast were harvested by centrifugation at 54 hours, filtered through 0.2 μm filters, and analyzed for ethanol, glucose, acetate and glycerol content by HPLC (Agilent Technology 1200 series) using Bio-Rad Aminex HPX-87H columns at 55° C., with an isocratic flow rate of 0.6 ml/min in 0.1 N H₂SO₄ eluent. 2.5 μl sample injection volumes were used. Calibration standards used for quantification including known amount of the analyses are shown in Table 1. Average ethanol increase is reported with reference to the FG strain.

TABLE 1
Analysis of fermentation broth following
fermentation for 54 hours at 32° C.

| Strain | Ethanol (g/l) | Glucose (g/l) | Increase in ethanol |
|---|---|---|---|
| FG | 146.1 | 0.46 | N/A |
| FG-mnn4 | 147.6 | 0.80 | 1.0% |

Yeast harboring the YKL201C gene deletion produce about 1.0% more ethanol compared to the unmodified reference strain at 32° C.

What is claimed is:

1. Modified yeast cells derived from parental yeast cells, the modified cells comprising a genetic alteration that causes the modified cells to produce a decreased amount of functional MNN4 polypeptide compared to the parental cells, wherein the modified cells produce during fermentation an increased amount of ethanol compared to parental cells under equivalent fermentation conditions.

2. The modified cells of claim 1, wherein the genetic alteration comprises a disruption of the YKL201C gene present in the parental cells.

3. The modified cells of claim 2, wherein disruption of the YKL201C gene is the result of deletion of all or part of the YKL201C gene.

4. The modified cells of claim 2, wherein disruption of the YKL201C gene is the result of deletion of a portion of genomic DNA comprising the YKL201C gene.

5. The modified cells of claim 2, wherein disruption of the YKL201C gene is the result of mutagenesis of the YKL201C gene.

6. The modified cells of any of claims 2-5, wherein disruption of the YKL201C gene is performed in combination with introducing a gene of interest at the genetic locus of the YKL201C gene.

7. The modified cells of any of claims 1-6, wherein the cells do not produce functional MNN4 polypeptides.

8. The modified cells of any of claims 1-6, wherein the cells do not produce MNN4 polypeptides.

9. The modified cells of any of claims 1-8, wherein the cells further comprise an exogenous gene encoding a carbohydrate processing enzyme.

10. The modified cells of any of claims 1-9, further comprising an alteration in the glycerol pathway and/or the acetyl-CoA pathway.

11. The modified cells of any of claims 1-10, further comprising an alternative pathway for making ethanol.

12. The modified cells of any of claims 1-11, wherein the cells are of a Saccharomyces spp.

13. A method for producing a modified yeast cell comprising: introducing a genetic alteration into a parental yeast cell, which genetic alteration reduces or prevents the production of functional MNN4 polypeptide compared to the parental cells, thereby producing modified cells that produces during fermentation an increased amount of ethanol compared to the parental cells under equivalent fermentation conditions.

14. The method of claim 13, wherein the genetic alteration comprises disrupting the YKL201C gene in the parental cells by genetic manipulation.

15. The method of claim 13 or 14, wherein the genetic alteration comprises deleting the YKL201C gene in the parental cells using genetic manipulation.

16. The method of any of claims 13-15, wherein disruption of the YKL201C gene is performed in combination with introducing a gene of interest at the genetic locus of the YKL201C gene.

17. The method of any of claims 13-16, wherein disruption of the YKL201C gene is performed in combination with making an alteration in the glycerol pathway and/or the acetyl-CoA pathway.

18. The method of any of claims 13-17, wherein disruption of the YKL201C gene is performed in combination with adding an alternative pathway for making ethanol.

19. The method of any of claims 13-18, wherein disruption of the YKL201C gene is performed in combination with disrupting the YJL065 gene present in the parental cells.

20. The method of any of claims 13-19, wherein disruption of the YKL201C gene is performed in combination with introducing an exogenous gene encoding a carbohydrate processing enzyme.

21. The method of any of claims 13-20, wherein the modified cell is from a Saccharomyces spp.

22. The method of any of claims 13-21, wherein the amount of ethanol produced by the modified yeast cells and the parental yeast cells is measured at 54 hours following inoculation of a hydrolyzed starch substrate comprising 34-35% dissolved solids and having a pH of 4.8.

23. Modified yeast cells produced by the method of any of claims 13-22.


SEQUENCE LISTING (2 sequences)

SEQ ID NO: 1 - protein, 1178 aa, Saccharomyces cerevisiae
MLQRISSKLHRRFLSGLLRVKHYPLRRILLPLILLQIIIITFIWSNSPQRNGLGRDADYLLPNYNELDSDDDSWYSILTSSFKNDRKIQFAKTLYENLKFGTNPKWVNEYTLQNDLLSVKMGPRKGSKLESVDELKFYDFDPRLTWSVVLNHLQNNDADQPEKLPFSWYDWTTFHELNKLISIDKTVLPCNFLFQSAFDKESLEAIETELGEPLFLYERPKYAQKLWYKAARNQDRIKDSKELKKHCSKLFTPDGHGSPKGLRFNTQFQIKELYDKVRPEVYQLQARNYILTTQSHPLSISIIESDNSTYQVPLQTEKSKNLVQSGLLQEYINDNINSTNKRKKNKQDVEFNHNRLFQEFVNNDQVNSLYKLEIEETDKFTFDKDLVYLSPSDFKFDASKKIEELEEQKKLYPDKFSAHNENYLNSLKNSVKTSPALQRKFFYEAGAVKQYKGMGFHRDKRFFNVDTLINDKQEYQARLNSMIRTFQKFTKANGIISWLSHGTLYGYLYNGMAFPWDNDFDLQMPIKHLQLLSQYFNQSLILEDPRQGNGRYFLDVSDSLTVRINGNGKNNIDARFIDVDTGLYIDITGLASTSAPSRDYLNSYIEERLQEEHLDINNIPESNGETATLPDKVDDGLVNMATLNITELRDYITSDENKNHKRVPTDTDLKDLLKKELEELPKSKTIENKLNPKQRYFLNEKLKLYNCRNNHFNSFEELSPLINTVFHGVPALIPHRHTYCLHNEYHVPDRYAFDAYKNTAYLPEFRFWFDYDGLKKCSNINSWYPNIPSINSWNPNLLKEISSTKFESKLFDSNKVSEYSFKNLSMDDVRLIYKNIPKAGFIEVFTNLYNSFNVTAYRQKELEIQYCQNLTFIEKKKLLHQLRINVAPKLSSPAKDPFLFGYEKAMWKDLSKSMNQTTLDQVTKIVHEEYVGKIIDLSESLKYRNFSLFNITFDETGTTLDDNTEDYTPANTVEVNPVDFKSNLNFSSNSFLDLNSYGLDLFAPTLSDVNRKGIQMFDKDPIIVYEDYAYAKLLEERKRREKKKKEEEEKKKKEEEEKKKKEEEEKKKKEEEEKKKKEEEEKKKKEEEEKKKQEEEEKKKKEEEEKKKQEEGEKMKNEDEENKKNEDEEKKKNEEEEKKKQEEKNKKNEDEEKKKQEEEEKKKNEEEEKKKQEEGHSN

SEQ ID NO: 2 - DNA, 3537 bp, Saccharomyces cerevisiae
atgcttcagcgaatatcatctaaacttcacaggcggttcttatctggcctgctgcgtgtcaagcactacccattaaggcgcattctccttccactgattctactgcagatcatcattataacgtttatctggtcaaattcaccgcagcgtaacggacttgggcgggacgctgattaccttctaccaaattacaacgaacttgacagtgatgatgattcctggtatagcatcctgacttcgtctttcaaaaacgatcgcaagatccagttcgctaagacattatacgaaaatttaaaattcggcaccaaccctaaatgggtcaatgaatatactctgcaaaatgacctgctctcggtcaaaatgggccctcgaaagggcagtaagctcgaatccgtggatgagttgaagttttacgacttcgaccctcgtctcacgtggtccgttgtgctgaaccatttgcaaaataatgacgcagatcagccagaaaagttacccttttcatggtacgactggacaaccttccacgagctgaataagctgatttccatagataaaactgttctgccctgcaattttcttttccagtccgctttcgacaaagagtctttagaggccattgagacagagctcggcgaacctttgttcctatacgaaagaccaaagtacgcgcagaaactgtggtacaaggccgctagaaaccaggacagaatcaaagactcaaaggaactaaaaaagcattgttccaagctattcactccagacgggcatggctctcctaagggtttaagatttaatacgcaatttcaaataaaggagctgtatgataaagttagacccgaagtttaccaattgcaggcaagaaactacattttgactacacagtcgcatccactatccatttccatcatcgaatcagataattccacgtatcaagtccccttgcaaactgaaaaatcaaaaaacttggtgcaatccggcctgttgcaggaatatattaatgataacattaattctacgaacaagagaaagaaaaataaacaggacgtagaattcaaccataacaggcttttccaggaattcgtcaataacgaccaagttaactccctatacaaactggaaattgaagaaactgataaattcacttttgataaagatttggtttatttatccccttcggatttcaagttcgatgcctccaaaaaaattgaagagttagaggaacagaagaaactctatccggacaaattttccgctcataatgagaattatctgaacagtttgaagaattccgtaaagacaagccctgcattgcaaagaaagttcttctatgaggctggtgccgtgaagcaatataaaggtatggggttccatcgtgacaagaggttcttcaatgttgatacattaatcaatgataaacaagaataccaggctagattgaactcaatgatcagaacattccaaaagtttactaaagccaacggcatcatatcttggttgtctcacggaacgctgtacggctatctttacaatggaatggctttcccttgggataacgatttcgacttgcaaatgcccattaagcatttacaattgctcagtcaatacttcaaccaatctcttatattggaagacccaagacagggtaatggacgttatttcctagacgtcagcgactccttgacagtaagaattaacggtaacggtaaaaacaatatcgatgcaagattcattgacgtcgacaccggcctttacattgatattaccggtctagctagcacttctgcccctagtagggattacttgaattcttatattgaagagcggttgcaagaggaacatttggatatcaataatatccctgaatcgaacggtgagaccgctactttgcccgacaaagtagatgatgggttagtcaatatggctacactaaacatcactgagctacgtgattacattaccagcgacgaaaataaaaatcataaaagagtccccactgatactgatttgaaagatcttttgaaaaaggaactggaagagttaccaaagtctaagaccattgaaaacaagttgaatcctaaacaaagatattttctcaacgaaaaacttaaactttacaattgtagaaacaaccattttaactcgttcgaggaactatctcccttaatcaatactgttttccatggtgtgccagcgttgattcctcacagacatacctactgcttgcacaatgaatatcatgtacctgatagatatgcatttgatgcttacaaaaatactgcttatttgcccgaatttagattttggttcgactatgacgggttaaagaaatgcagtaatattaattcatggtatccaaacatccccagtattaattcatggaatccgaacctcttgaaagaaatatcgtctacgaaatttgagtcgaaactttttgattccaacaaagtctctgaatactctttcaaaaacctatccatggatgatgttcgcttaatttataaaaatattccaaaagctggctttatcgaggtatttactaacttgtacaattccttcaatgtcactgcatataggcaaaaggaattggaaattcaatactgccaaaacctgacatttattgaaaaaaagaaattattacatcaattgcgcattaatgttgctcctaagttaagctcccctgcaaaggacccatttctttttggttatgaaaaagctatgtggaaggatttatcaaaatctatgaaccagactacattagatcaagttaccaagattgttcatgaagaatatgtcggaaaaattattgatctgtccgaaagtttgaaatacaggaatttttcacttttcaacattacttttgatgaaactggaacaactctagatgataacacagaagattatactcctgctaatactgttgaagtaaatcctgtggattttaaatcaaatttaaactttagtagcaactcctttttggatttaaattcatatggtttagacctttttgcgccaactttatccgacgttaacagaaagggtattcaaatgtttgataaggaccctattattgtatacgaggactatgcttatgccaagttacttgaagaaagaaagcggagggagaagaagaagaaggaggaagaggagaagaagaagaaggaagaagaggaaaagaagaagaaggaagaagaagaaaagaaaaagaaggaagaggaagagaagaaaaagaaggaagaagaagagaagaaaaagaaggaagaagaagaaaagaagaagcaggaggaagaggagaaaaagaagaaggaagaagaagagaagaagaagcaggaagaaggagaaaagatgaagaatgaagatgaagaaaataagaagaatgaagatgaagaaaagaagaagaacgaagaagaggaaaaaaagaagcaggaagagaaaaacaagaagaatgaagatgaagaaaagaagaagcaggaagaggaagaaaagaagaagaacgaagaagaggaaaaaaagaagcaggaggaggggcacagcaattaa